(19) United States
(12) Patent Application Publication
(10) Pub. No.: US 2008/0249198 A1
(43) Pub. Date: Oct. 9, 2008

(54) Oxidation of Methane to Methanol using a Bimetallic Catalyst

(76) Inventors: Wensheng Chen, Hoffman Estates, IL (US); Simon R. Bare, Glen Ellyn, IL (US); Maureen L. Bricker, Buffalo Grove, IL (US); Timothy A. Brandvold, Arlington Heights, IL (US); Joseph A. Kocal, Glenview, IL (US)

Correspondence Address: HONEYWELL INTELLECTUAL PROPERTY INC;PATENT SERVICES, 101 COLUMBIA DRIVE, P O BOX 2245 MAIL STOP AB/2B, MORRISTOWN, NJ, 07962, US

(21) Appl. No.: 11/697,780

(22) Filed: Apr. 9, 2007

(51) Int. Cl. C07C 27/06

(52) U.S. Cl. 518/703

(57) ABSTRACT

A process for the oxidation of methane to methanol has been developed. The process involves contacting a gas stream, comprising methane, a solvent and an oxidizing agent with a bimetallic catalyst at oxidation conditions to produce a methyl ester. Finally, the methyl ester is hydrolyzed to yield a methanol product stream. The bimetallic catalyst comprises at least two transition metal components. One example of the catalytic component is a combination of cobalt and manganese.

### FIELD OF THE INVENTION

This invention relates to a process for converting methane to methanol using a bimetallic catalyst comprising a combination of at least two transition metal components. Generally the process involves contacting a gas stream, comprising methane, a solvent and an oxidizing agent such as air with the catalyst at oxidation conditions to produce a methyl ester. Finally, the methyl ester is hydrolyzed to yield a methanol product stream.

### BACKGROUND OF THE INVENTION

Today, both chemical and energy industries rely on petroleum as the principal source of carbon and energy. Methane is underutilized as a chemical feedstock, despite being the primary constituent of natural gas, an abundant carbon resource. Factors limiting its use include the remote locations of known reserves, its relatively high transportation costs and its thermodynamic and kinetic stability. Methane's main industrial use is in the production of synthesis gas or syngas via steam reforming at high temperatures and pressures. Syngas in turn can be converted to methanol also at elevated temperatures and pressures. The production of methanol is important because methanol can be used to produce important chemicals such as olefins, formaldehyde, acetic acetate, acetate esters and polymer intermediates. The above two step process for the production of methanol is expensive and energy intensive with corresponding environmental impacts.

Selective oxidation of methane has been studied for over 30 years by individual, academic and government researchers with no commercial success. For example, Sen et al. in New J Chem, 1989, 13, 755-760 disclose the use of Pd(O₂C Me)₂ in trifluoroacetic acid for the oxidation of methane to CF₃CO₂Me. The reaction is carried out for 4 days at a pressure of 5516-6895kPa (800-1000 psi). E. D. Park et al. in Catalysis Communications, Vol. 2 (2001), 187-190, disclose a Pd/C plus Cu(CH₃COO)₂ catalyst system for the selective oxidation of methane using H₂/O₂ to provide H₂O₂ in situ. L. C. Kao et al. in JAm. Chem.Soc., 113 (1991), 700-701 disclose the use of palladium compounds such as Pd(O₂CC₂H₅)₂ to oxidize methane to methanol in the presence of H₂O₂ and using trifluoroacetic acid as the solvent. U.S. Pat. No. 5,585,515 discloses the use of catalysts such as Cu(I) ions in trifluoroacetic acid to oxidize methane to methanol. WO 2004069784 A1 discloses a process for the oxidation of methane to methanol using transition metals such as cobalt or manganese in trifluoroacetic acid. Finally, M. N. Vargaftik et al in J Chem. Soc., Chem. Commun. 1990(15) pp. 1049-1050 disclose results for a number of metal perfluoro acetate compounds. The metals which were found to be active were Pd, Mn, Co and Pb. Copper was found to have virtually no activity.

Applicants have developed a process which uses a bimetallic catalyst. The bimetallic catalyst comprises at least two transition metal components such as cobalt and manganese. Methane, a solvent such as trifluoroacetic acid and an oxidizing agent such as air are contacted with the catalyst at oxidation conditions to provide a methyl ester. The methyl ester, e.g. methyl trifluoroacetate, is subsequently hydrolyzed to give a methanol stream.

### SUMMARY OF THE INVENTION

As stated, this invention relates to a process for converting methane to methanol comprising contacting a gas stream comprising methane with a bimetallic catalyst comprising a combination of at least two transition metal components, in the presence of an oxidizing agent and a solvent at oxidation conditions to provide a methyl ester compound and hydrolyzing the methyl ester compound at hydrolysis conditions to provide a methanol product stream. One example of the transition metal components is manganese and cobalt, while an example of a solvent is trifluoroacetic acid.

Additional objects, embodiments and details of this invention can be obtained from the following detailed description of the invention.

### DETAILED DESCRIPTION OF THE INVENTION

The present invention relates to a process for the oxidation of methane to methanol. One necessary component of the invention is a bimetallic catalyst comprising a combination of at least two transition metal components. The transition metals are selected from the group consisting of manganese, silver, cobalt, mercury, palladium, lead, platinum, iron, molybdenum, copper, and vanadium. Specific combinations of metals include without limitation manganese and silver, manganese and cobalt, manganese and iron, manganese and mercury, silver and cobalt, copper and manganese, and molybdenum and vanadium.

The transition metals can be used in any form which is active in catalyzing the selective oxidation of methane to methanol. The transition metal compounds which can be used include without limitation, metal oxides, metal salts, organometallic compounds, etc. Specific examples of the transition metal compounds include without limitation Mn₂O₃, Mn₃O₄, MnO₂, KMnO₄, K₂Mn₄P₃O₁₆, MnPO₄.H₂O, Na₂Mn₂P₂O₉.H₂O, KMn₈O₁₆, (FeMn)PO₄, Mn(II)trifluoroacetate, Mn(II)acetate, Mn(III)acetate, Co₂O₃, Co(II)Acetate, AgO, Ag(I)trifluoroacetate, Fe₂O₃, etc. The amount of each metal present in the reaction mixture, i.e. solvent plus oxidant plus methane can vary from about 0.01 to about 10 wt. % as the metal.

In addition to the transition metal components being added to the solvent, they can be deposited onto a support. The supports which can be used include but are not limited to aluminas, silica, silicon carbide, silica-alumina, molecular sieves, ceria, zirconia, titania, magnesium oxide, lanthanum oxide, aluminum phosphate etc. It should be pointed out that silica-alumina is not a physical mixture of silica and alumina but means an acidic and amorphous material that has been cogelled or coprecipitated. This composition is well known in the art, see e.g. U.S. Pat. No. 3,909,450; U.S. Pat. No. 3,274,124 and U.S. Pat. No. 4,988,659 all of which are incorporated by reference in their entirety. Molecular sieves include zeolites and non-zeolitic molecular sieves (NZMS). Examples of zeolites include, but are not limited to, zeolite Y, zeolite X, zeolite L, zeolite beta, ferrierite, MFI, mordenite and erionite. Non-zeolitic molecular sieves (NZMS) are those molecular sieves which contain elements other than aluminum and silicon and include silicoaluminophosphates (SAPOs) described in U.S. Pat. No. 4,440,871, ELAPOs described in U.S. Pat. No. 4,793,984, MeAPOs described in U.S. Pat. No. 4,567,029 all of which are incorporated by reference. Aluminas include without restriction gamma alumina, delta alumina, eta alumina and theta alumina.

If the transition metal compounds are soluble they can be deposited onto the support by methods well known in the art which include without limitation impregnation, precipitation, etc. A preferred method is impregnation which is carried out by preparing a solution of the transition metal compounds and then contacting the support with the solution for a time sufficient to absorb the transition metal compound onto the support. The transition metal compounds which can be used to prepare the solution include without limitation the hydroxide, nitrate, acetate, chloride, oxalate, acetylacetonate (specific examples are enumerated above). In addition transition metal complexes which contain neutral or charged coordinating ligands can also be used. Water is the solvent which is usually used to prepare the solution although organic solvents such as ethanol or acetone can be used. Once the compound is absorbed onto the support, it is dried and then calcined at a temperature of about 100° C. to about 800° C. for a time of about 1 hour to about 48 hours. Depending on post synthesis treatment conditions the metal may be present on the support as a metal cation, metal oxide, reduced metal, or a mixture thereof. Regardless of the form of the transition metal on the support, each of the transition metals is present in an amount from about 0.1 wt. % to about 10 wt. % of the catalyst as the metal.

The catalyst comprising the support and bimetallic component can be used in the form of a powder or a shaped article. Examples of shaped articles include without limitation spheres, pills, pellets, extrudates, irregularly shaped particles, etc. Means for preparing these shaped articles are well known in the art. If the transition metal compounds are deposited onto the support by impregnation, deposition of the transition metal compounds can be done either before or after the powder is formed into a shaped article although not necessarily with equivalent results. Metal impregnation before forming is preferred. When the transition metal oxides or other compounds which are insoluble in an impregnation solvent are desired, they can be deposited on a support by commingling it with the support and then forming it into a shaped article by means such as extrusion, marumerizing, pelletizing, etc.

The oxidation of methane to methanol can be carried out in a batch process or a continuous process. In a batch process, the catalyst is placed into a reactor, to which is added a solvent followed by the addition of methane. Non limiting examples of solvents include trifluoroacetic acid, trifluoroacetic anhydride, pentafluoropropionic acid, acetic acid, sulfuric acid, sulfur trioxide, trifluoromethanesulfonic acid, methanesulfonic acid and super critical carbon dioxide with trifluoroacetic acid being preferred. To the mixture of catalyst and solvent is added methane in a concentration to produce a pressure of about 103 kPa (15 psi) to about 6895 kPa (1000 psi) and preferably from about 4137 kPa (600 psi) to about 6895 kPa (1000 psi). In addition to methane, catalyst and solvent, an oxidizing agent is necessary to carry out the reaction. Air is the usual oxidizing agent, although pure oxygen can be used, as well as synthetic blends containing oxygen and an inert diluent gas such as nitrogen, argon, helium, etc. Atmospheric air contains approximately 21% oxygen as a mixture with 78% nitrogen, and less than 1% carbon dioxide, water, and other trace gases. If air or other gaseous oxidizing agent is used, then the oxidizing agent is typically added to the reaction mixture directly from a compressed gas cylinder or tank or via atmospheric source with a mechanical compressor. The concentration of oxidizing agent can vary from about 0.1 mole % to about 50 mole %. The pressurized reaction vessel is now heated at a temperature of about 25° C. to about 250° C. and preferably from about 60° C. to about 100° C. The vessel is held at this temperature for a time of about 1 minute to about 24 hours in order to contact the methane with the oxidizing agent, catalyst and solvent and provide a mixture comprising a methyl ester formed from the methane and an adduct from the solvent.

The methyl ester formed, such as methyl trifluoroacetate, can be separated from the reaction mixture by any suitable methods but distillation is preferred. The methyl ester, e.g. methyl trifluoroacetate (MTFA) is now hydrolyzed to produce free methanol and regenerate the solvent. Using MTFA as an example, although it is understood that the process is not limited to MFTA, the MFTA is introduced into a hydrolysis reactor along with water. The amount of water introduced is at least the stoichiometric amount required for complete hydrolysis although it is preferred to use an excess amount of water. A catalyst and a co-solvent may also be used. A variety of acidic and basic substances are known to promote ester hydrolysis. Suitable acids include but are not limited to hydrochloric acid, sulfuric acid, trifluoroacetic acid, toluene sulfonic acid, acidic alumina, silica-alumina, sulfated zirconia, and acidic ion exchange resins, and suitable basic materials include but are not limited to sodium hydroxide, lithium hydroxide, potassium hydroxide, and solid bases such as hydrotalcite. Acid hydrolysis is preferred to allow easy recovery of the trifluoroacetic acid solvent/product. When hydrolysis is complete the methanol product can be separated from the reaction mixture by a variety of methods known in the art including distillation, adsorption, extraction and diffusion through a membrane. Separation of trifluoroacetic acid is achieved by analogous methods. The recovered trifluoroacetic acid is then recycled to the oxidation reactor.

In addition to carrying out the process in a batch mode as described above, the process can also, be conducted in a continuous mode as follows. The catalyst is placed in a fixed bed high pressure reactor and the methane, oxidizing agent and solvent flowed through the bed at the temperatures and pressures set forth above. Methane, oxidizing agent and solvent may be added independently to the reactor or mixed prior to introduction to the reactor. The solvent/methane/oxidizing agent mixture is flowed through the catalyst bed at a liquid hourly space velocity (LHSV) of about 0.1 hr⁻¹ to about 100 hr⁻¹. Gas and liquid are removed from the reactor continuously at a rate to maintain the liquid level and total pressure in the reactor. The removed gas/liquid stream is transferred to a vessel where the gas and liquid are separated and one or both streams may be subjected to further separation or returned to the high pressure reactor.

### EXAMPLES 1-25

A series of experiments were conducted to investigate the activity of various bimetallic catalysts at various temperatures and with and without added oxygen. The general procedure is set forth below and the results are presented in The Table. To an 80 cc Parr™ reactor there were added 10 ml of trifluoroacetic acid and 300 mg of a first catalyst and 20 mg of an additive catalyst. The reactor was assembled and pressurized first with methane to 4238 kPa (600 psig) and if oxygen was added, the reactor was further pressurized with 2758 kPa (400 psig) of 8% oxygen in nitrogen. The reactor was heated to various temperatures for 3 hours. The liquid sample was analyzed by GCMS and the gas sample analyzed by GC equipped with FID, TCD and MS detectors. The estimated methane based yield was calculated based on methanol product (isolated as methyl trifluoroacetate) divided by methane introduced into the system. Methanol product was calculated based on GCMS analysis, and the amount of methane introduced into the system was based on the weight difference before and after the introduction of methane gas and ideal gas law occasionally.

THE TABLE
Effect of Catalyst and Oxidant on Methane to Methanol Production

| | | Additive | Additional | Temperature | Methanol |
|---|---|---|---|---|---|
| Run | Catalyst | Catalyst | Oxidant | (° C.) | Yield (%)* |
| 1 | Mn2O3 | — | None | 180 | 1.97% |
| 2 | Mn2O3 | — | None | 160 | 2.04% |
| 3 | Mn2O3 | — | None | 140 | 1.32% |
| 4 | MnO2 | — | None | 180 | 1.04% |
| 5 | MnO2 | — | None | 160 | 0.60% |
| 6 | MnO2 | — | None | 140 | 0% |
| 7 | Mn(TFA)2 | — | 2758 kPa¹ | 180 | 1.70% |
| 8 | Mn(TFA)2 | — | 2758 kPa¹ | 160 | 1.01% |
| 9 | Mn(TFA)2 | — | 2758 kPa¹ | 140 | 0% |
| 10 | Mn2O3 | Cu(TFA)2 | None | 180 | 1.75% |
| 11 | Mn2O3 | Cu(TFA)2 | None | 160 | 2.01% |
| 12 | Mn2O3 | Cu(TFA)2 | None | 140 | 2.29% |
| 13 | Mn2O3 | Cu(TFA)2 | None | 120 | 1.53% |
| 14 | Mn2O3 | Cu(TFA)2 | None | 110 | 1.58% |
| 15 | Mn2O3 | Cu(TFA)2 | None | 105 | 0.53% |
| 16 | Mn2O3 | Co3O4 | None | 180 | 1.59% |
| 17 | Mn2O3 | Co3O4 | None | 160 | 1.79% |
| 18 | Mn2O3 | Co3O4 | None | 140 | 1.88% |
| 19 | Mn2O3 | Co3O4 | None | 120 | 1.09% |
| 20 | Mn2O3 | Co3O4 | None | 110 | 1.26% |
| 21 | Mn2O3 | Co3O4 | None | 105 | 1.33% |
| 22 | Mn2O3 | Pd(TFA)2 | None | 180 | 1.47% |
| 23 | Mn2O3 | Pd(TFA)2 | None | 160 | 1.11% |
| 24 | Mn2O3 | Pd(TFA)2 | None | 140 | 1.15% |
| 25 | Mn2O3 | Pd(TFA)2 | None | 120 | 1.12% |

*Yield based on total methane added into the reactor.
¹Oxidant is 8% oxygen in nitrogen.

1. A process for converting methane to methanol comprising contacting a gas stream comprising methane with a bimetallic catalyst comprising a combination of at least two transition metal components in the presence of an oxidizing agent selected from the group consisting of oxygen, air and mixtures thereof and a solvent at oxidation conditions to provide a methyl ester compound and hydrolyzing the methyl ester compound at hydrolysis conditions to provide a methanol product stream.

2. The process of claim 1 where the oxidation conditions comprise a temperature of about 80° C. to about 200° C., a pressure of about 103kPa (15 psia) to about 6867kPa (1000 psia), a contact time of about 1 min to about 24 hrs and an oxidizing agent concentration from about 0.1 mol % to about 50 mol %.

3. The process of claim 1 where the hydrolysis conditions include a temperature of about 20° C. to about 200° C. and a pressure of about 103kPa (15psi) to about 1030kPa (150psi) and at least a stoichiometric amount of water.

4. The process of claim 1 further comprising carrying out the hydrolysis in the presence of a catalyst selected from the group consisting of acidic catalysts and basic catalysts.

5. The process of claim 4 where the acidic catalyst is selected from the group consisting of hydrochloric acid, sulfuric acid, trifluoroacetic acid, toluene sulfonic acid, acidic alumina, silica-alumina, sulfated zirconia, acidic ion exchange resins and mixtures thereof.

6. The process of claim 4 where the basic catalyst is selected from the group consisting of sodium hydroxide, lithium hydroxide, potassium hydroxide and hydrotalcite.

7. The process of claim 1 where the transition metal components are at least two metals selected from the group consisting of manganese, silver, cobalt, mercury, palladium, lead, platinum, iron, molybdenum, copper, and vanadium.

8. The process of claim 1 where the transition metal component is present as the metal oxides, metal salts, organometallic compounds or mixtures thereof.

9. The process of claim 8 where the transition metal component is selected from the group consisting of Mn₂O₃, Mn₃O₄ MnO₂, KMnO₄, K₂Mn₄P₃O₁₆, MnPO₄, H₂O, Na₂Mn₂P₂O₉H₂O, KMn_{g}O₁₆, Mn(II)trifluoroacetate, Mn(II) acetate, Mn(III)acetate, Co₂O₃, Co(II) Acetate, AgO, Ag(I)trifluoroacetate, Fe₂O₃, (FeMn)PO₄ and mixtures thereof.

10. The process of claim 1 where the transition metal component is deposited onto an inorganic oxide support.

11. The process of claim 10 where the inorganic oxide is selected from the group consisting of aluminas, silica, silica-alumina, molecular sieves, ceria, zirconia, titania, magnesium oxide, lanthanum oxide, aluminum phosphate and mixtures thereof.

12. (canceled)

13. The process of claim 1 where the oxidizing agent is intermittently added.

14. The process of claim 1 where the solvent is selected from the group consisting of trifluoroacetic acid, trifluoroacetic anhydride, pentafluoropropionic acid, acetic acid, super critical carbon dioxide, sulfuric acid, sulfur trioxide, trifluoromethanesulfonic acid, methanesulfonic acid and mixtures thereof.

15. The process of claim 1 where the process is a batch process.

16. The process of claim 1 where the process is a continuous process.

17. The process of claim 1 where the oxidizing agent is air.

18. The process of claim 1 where the oxidizing agent is oxygen blended with an inert diluent selected from the group consisting of nitrogen, argon, helium and mixtures thereof.
